(19) United States
(12) Patent Application Publication
(10) Pub. No.: US 2008/0064758 A1
(43) Pub. Date: Mar. 13, 2008

(54) METHOD OF TREATMENT OR PROPHYLAXIS OF DEPRESSION

(75) Inventors: Kenneth W. Locke, Carlsbad, CA (US); Richard E. Gammans, Vass, NC (US)

Correspondence Address: FOLEY AND LARDNER LLP;SUITE 500, 3000 K STREET NW, WASHINGTON, DC, 20007, US

(73) Assignee: MediciNova, Inc.

(21) Appl. No.: 11/842,384

(22) Filed: Aug. 21, 2007

Related U.S. Application Data
(60) Provisional application No. 60/823,323, filed on Aug. 23, 2006.
(60) Provisional application No. 60/893,266, filed on Mar. 6, 2007.

(51) Int. Cl. A61K 31/135; A61P 43/00

(52) U.S. Cl. 514/658

(57) ABSTRACT

The invention concerns a method of treatment or prophylaxis of depression by administering a phenylethanolaminocyclohexylphenyl compound of a general formula (I) 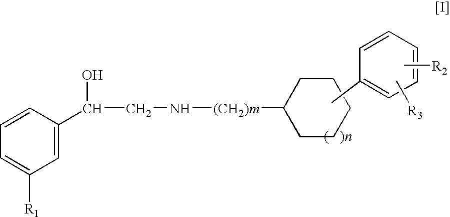
in which R₁ represents hydrogen or halogen; R₂ represents hydrogen, hydroxy, lower alkoxy, lower alkoxy substituted with one or two lower alkoxycarbonyl or carboxy groups, lower alkoxy substituted with lower alkylaminocarbonyl which may be substituted with lower alkoxy, lower alkoxy substituted with cyclic aminocarbonyl of 4 to 6 carbon atoms, lower alkoxycarbonyl or carboxy; R₃ represents hydrogen, hydroxy, lower alkoxy or lower alkoxy substituted with one or two lower alkoxycarbonyl or carboxy groups; R₂ and R₃ may be bonded to each other to form methylenedioxy substituted with carboxy or lower alkoxycarbonyl; and m and n are 0 or 1, and their pharmacologically acceptable metabolites or salts, which have selective stimulating activity and binding affinity for β₃ adrenergic receptors.

### CROSS-REFERENCE TO RELATED APPLICATIONS

The subject application claims priority to U.S. Provisional Application No. 60/823,323 filed Aug. 23, 2006 and U.S. Provisional Application No. 60/893,266 filed Mar. 6, 2007, the disclosures of which are incorporated herein by reference in their entirety.

### SUMMARY OF THE INVENTION

The present invention concerns the use of phenylethanolaminocyclohexylphenyl compounds, preferably phenylethanolaminocyclohexylphenoxyacetic acid ester compounds and their metabolites and salts, for the treatment or prophylaxis of depression. In a preferred embodiment of the invention a method is described for treating or preventing depression which comprises administering to a mammal in need thereof a therapeutically or prophylactically effective amount of at least one compound of a general formula (I) 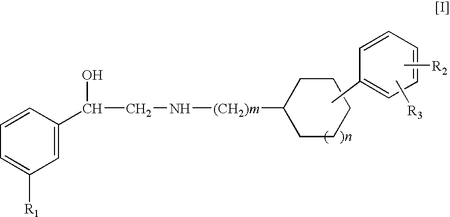
in which R₁ represents hydrogen or halogen; R₂ represents hydrogen, hydroxy, lower alkoxy, lower alkoxy substituted with one or two lower alkoxycarbonyl or carboxy groups, lower alkoxy substituted with lower alkylaminocarbonyl which may be substituted with lower alkoxy, lower alkoxy substituted with cyclic aminocarbonyl of 4 to 6 carbon atoms, lower alkoxycarbonyl or carboxy; R₃ represents hydrogen, hydroxy, lower alkoxy or lower alkoxy substituted with one or two lower alkoxycarbonyl or carboxy groups; R₂ and R₃ may be bonded to each other to form methylenedioxy substituted with carboxy or lower alkoxycarbonyl; and m and n are 0 or 1, and their pharmacologically acceptable metabolites or salts, which have a potent β₃ adrenergic stimulating effect (β₃ agonist) and high β₃ adrenergic receptor selectivity.

Compounds of a general formula (I) and their methods of preparation are known in the prior-art. See, for example, U.S. Pat. No. 6,069,176 granted to Tsuchiya et al., the complete disclosure of which is incorporated by reference herein. Compounds of a general formula (I) are known for treating a number of ailments in animals, including humans, such as accelerated or spasmodic gastrointestinal motility, dysuria, pollakisuria, urinary incontinence, obesity and diabetes. These compounds have not heretofore been shown to exhibit effectiveness in the treatment or prophylaxis of depression.

It has now been found that compounds of a general formula (I) which possess a selective stimulating activity and binding affinity for β₃ adrenergic receptors are therapeutically very interesting as anti-depressant agents, as they have an anti-depressant activity at least comparable to classical antidepressant agents (e.g., monoamine oxidase inhibitors, tricyclic antidepressants, selective serotonin and/or norepinephine reuptake inhibitors). By “depression” is meant generically any condition falling under either or both categories of Mood Disorders and depression, including but not limited to Major Depressive Episodes. Examples of conditions that can be considered suitable for treatment and/or prophylaxis include but are not limited to Major Depressive Disorder, Dysthymia, Depression NOS, Depression due to a medical condition, Bipolar I and II, Mood Disorder due to a medical condition, Substance-Induced Mood Disorder, Mood Disorder NOS and the like.

### DETAILED DESCRIPTION OF THE PREFERRED EMBODIMENTS

A preferred embodiment of the invention is Compound A, depicted below, which is ethyl 3-[(1R,3R)-3-[(2R)-2-(3-chlorophenyl)-2-hydroxyethylamino]cyclohexyl]-phenoxyacetate or a pharmaceutically acceptable metabolite or salt thereof. In particular, Compound A is 3-[(1R,3R)-3-[(2R)-2-(3-chlorophenyl)-2-hydroxyethylamino]cyclohexyl]-phenoxy acetic acid ethyl ester, maleate salt (CA Index Name: Acetic acid, [3-[(1R,3R)-3-[[(2R)-2-(3-chlorophenyl)-2-hydroxyethyl]amino]cyclohexyl]phenoxy]-, ethyl ester, (2Z)-2-butenedioate (1:1) (salt); CAS Registry Number: 863514-91-4).

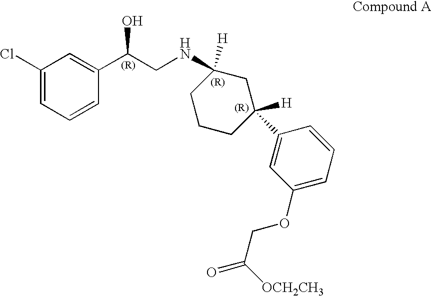

An example of a suitable metabolite thereof is the ethyl ester hydrolysis product, 3-[(1R,3R)-3-[(2R)-2-(3-chlorophenyl)-2-hydroxyethylammonium]cyclohexyl]phenoxyacetate, the chemical formula for which is depicted below. An example of a suitable salt thereof is an acid addition salt, such as a maleate salt, or a metal salt, such as a sodium, potassium, magnesium, or calcium salt or the like.

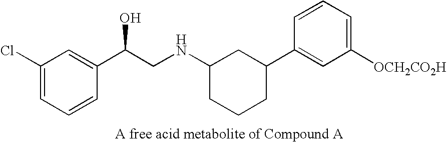

The anti-depressant properties of compounds of a general formula (I) are studied by means of a set of tests in animals conventionally employed in pharmacology and generally considered predictive of anti-depressant activity in man (e.g, P. Simon, Therapie (1973) 28:209-223). More particularly compounds of a general formula (I) are tested in rodents in comparison with compounds with known clinical antidepressant properties in the tests including, but not limited to those described below:

Antagonism of Apomorphine-Induced Hypothermia (A. J. Puech et al., Psychopharmacology (1981) 75(1):84-91)

Male CD1 mice (Charles River) weighing 22-25 g are used in the test. The mice are housed individually in transparent plastic cages. The compounds of a general formula (I) as well as the reference compounds are suspended in 1% aqueous carboxymethylcellulose and administered intraperitoneally (10 ml/kg; 6 mice per dose). Control animals received the vehicle only.

Rectal temperature is measured using a probe carrying a thermoelectric couple (connected to a galvanometer) inserted to a constant depth.

The compounds to be tested as well as the vehicle are administered 30 minutes after basal rectal temperature is measured, and 30 minutes later apomorphine (16 mg/kg) is administered subcutaneously. Rectal temperature is measured again 30 minutes after the administration of apomorphine.

The results are expressed as average rectal temperature of each group, by evaluating the significant differences in temperature between treated and control mice by Student's t test, and are used for calculating the M.A.D., i.e., the minimal active dose. The results suggest M.A.D. (mg/kg i.p.) values for compounds of a general formula (I) which are intermediate to clenbuterol (0.03 mg/kg i.p.) and salbutamol (1.0 mg/kg i.p.).

Antagonism of the Reserpine-Induced Hypothermia

The compounds of a general formula (I) as well as the reference compounds are administered 4 hours after reserpine (2.5 mg/kg i.p.). Rectal temperature is measured 90 minutes after the administration of the compounds to be tested. The results obtained with Compound A, as a representative compound of a general formula (I), suggest a M.A.D. (mg/kg i.p.) value that is closer to clenbuterol (0.03 mg/kg i.p.) than salbutamol (3.0 mg/kg i.p.).

Antagonism of the Oxotremorine-Induced Hypothermia

The test compounds are administered 30 minutes before oxotremorine (0.4 mg/kg i.p.). Rectal temperature is measured 30 minutes after the administration of oxotremorine. The oxotremorine-induced hypothermia is antagonised, at least partially, by the compounds of a general formula (I) as well as by the reference compounds. With Compound A, whose activity in this test will be investigated more deeply, it is expected that antagonistic activity will increase in a regular manner with the dose.

Potentiation of Yohimbine Toxicity

Yohimbine administration (30 mg/kg s.c.) generally causes the death of one out of ten mice. Potentiation of this toxicity by the test compounds is evaluated by administering the test compounds intraperitoneally 30 minutes before yohimbine administration and calculating the number of deaths in the 24 hours following yohimbine s.c. injection. The compounds of a general formula (I), which are submitted to this test, are expected to show a clear potentiation of yohimbine toxicity comparable to or in excess of that shown by the reference compounds.

A protective or ameliorating effect against depression is demonstrated also when the compounds of a general formula (I) are administered intracranially at very low doses, which definitely exclude any peripheral effect on colon. The central anti-depressant effects elicited by the compounds of a general formula (I) are similar to those obtained with the reference compounds.

Tail Suspension

Compounds of a general formula (I) (starting at an initial dose of 30 mg/kg PO, 1 hr pretreatment) were administered to mice (n=5). A reduction of immobility by 50% or more relative to a vehicle-treated control group indicates possible antidepressant activity. Fluoxetine hydrochloride served as a standard reference compound. The mice displayed an inverted U-shaped dose-response curve, which is not an unusual response in anti-depressant assays. There was a 35% mobility reduction at 100 mg/kg PO, 54% mobility reduction at 10 mg/kg PO and 46% mobility reduction at 1 mg/kg PO. The results are suggestive of antidepressant activity. The fluoxetine reference demonstrated 87% mobility reduction at 30 mg/kg PO.

Adenylyl Cyclase Assay

MN-246 was tested at full-log concentrations ranging from 1 nM to 10 μM for agonism/antagonism in the β3 adrenergic cellular adenylyl cyclase assay in HEK 293 cells and at the same concentrations for potential cytotoxicity in the corresponding β3 adrenergic cytotoxicity assay. Cells were incubated for 20 minutes at 37° C. in Hank's buffered salt solution, with 1 mM MgCl₂, 1 mM CaCl₂, at a pH of 7.4. Enzyme immunoassay (EIA) was used to measure cAMP accumulation. The compound MN-246 caused agonist-like stimulation of adenylyl cyclase activity with an estimated EC₅₀ of 76 nM. No cytotoxicity was observed at any test concentration.

Tetrabenazine Methane Sulfonate (TBZ) Hypothermia

Compounds of a general formula (I) (starting at an initial dose of 30 mg/kg PO) are administered to mice (n=5) 30 minutes after exposure of the mice to TBZ (85 mg/kg i.p.). A reduction by 50% or more relative to a vehicle-treated control group of TBZ-induced hypothermia 90 minutes after administration of test compound indicates possible antidepressant activity. Imipramine serves as a standard reference compound.

Furthermore, the compounds of a general formula (I) and their pharmaceutically acceptable metabolites and salts have a very low toxicity which is compatible with their use as drugs.

In view of the above a first object of the present invention is therefore a method of treatment or prophylaxis of depression by administering a therapeutically or prophylactically effective dose of at least one compound of a general formula (I) or of a pharmaceutically acceptable metabolite or salt thereof.

A preferred object of the present invention is a method of treatment or prophylaxis of depression which comprises administering to a mammal in need thereof an antidepressant effective dose of at least one compound of a general formula (I) or a pharmaceutically acceptable metabolite or salt thereof.

In a preferred embodiment of the invention an RI represents halogen, an R₂ represents lower alkoxy substituted with one or two lower alkoxycarbonyl groups, R₃ represents hydrogen, and in which m is 0 and n is 1. A preferred compound of a general formula (I) is ethyl 3-[(1R,3R)-3-[(2R)-2-(3-chlorophenyl)-2-hydroxyethylamino]-cyclohexyl]phenoxyacetate. A pharmaceutically acceptable metabolite thereof is the ethyl ester hydrolysis product, 3-[(1R,3R)-3-[(2R)-2-(3-chlorophenyl)-2-hydroxyethyl-ammonium]cyclohexyl]phenoxyacetate.

The at least one compound of a general formula (I) or a pharmaceutically acceptable metabolite or salt thereof can be administered in a daily dosage ranging from about 0.01 to about 30 mg/kg, preferably ranging from about 0.01 to about 10 mg/kg.

More generally, the present invention is directed to a treatment or prophylaxis of depression which comprises administering to a mammal in need thereof a therapeutically or prophylactically effective amount of at least one phenylethanolaminocyclohexylphenoxyacetic acid ester compound or a pharmacologically acceptable metabolite or salt thereof. Such compounds preferably exhibit selective stimulating activity and binding affinity for β₃ adrenergic receptors. More preferably, such compounds are chosen in which one or more, two or more, three or more, or all the chiral centers present in the molecule possess a stereochemical configuration (R).

As used herein, the term pharmaceutically acceptable salts includes the acid addition salts of pharmaceutically acceptable mineral or organic acids as well as the salts of the compounds of a general formula (I) with mineral bases, preferably those with alkali metals such as sodium or potassium or alkaline-earth metals such as magnesium or calcium, or with pharmaceutically acceptable organic bases, such as tartaric acid, maleic acid and the like.

Among the acids which can be employed for the salification of the free base, there may be cited as examples: hydrochloric acid, hydrobromic acid, acetic acid, formic acid, propionic acid, oxalic acid, fumaric acid, maleic acid, succinic acid, benzoic acid, cinnamic acid, mandelic acid, citric acid, malic acid, tartaric acid, aspartic acid, glutamic acid, methanesulfonic acid, p-toluenesulfonic acid, and the like.

In the above formula for Compound A, the three asymmetric carbons are marked by a designation (R). However, all the compounds of a general formula (I) may exist in at least six stereoisomeric forms: (R,R,R), (R,R,S), (R,S,S), (S,S,S), (S,S,R), and (S,R,R).

The optically pure isomers, as well any mixture of two, three, four, five, or all six isomers, in any proportion, may be utilized for the methods of treatment and prophylaxis according to the present invention. It is however understood that, as it often happens when dealing with pharmaceutically active compounds possessing one or more chiral centers, the different stereoisomers may have different activity levels. If desired, the person skilled in the art may, on the basis of the indications given in the present application and his own experience, choose from the different stereoisomers of a general formula (I) the compound or compounds that are therapeutically most effective.

In a preferred embodiment of the invention, compounds of a general formula (I) wherein the carbon atom linked to the hydroxyl group has an absolute configuration (R) are generally more interesting. Hence, the use of at least one compound of a general formula (I) wherein the carbon atom linked to the hydroxyl group has an absolute configuration (R) in a method of treating depression represents a preferred embodiment of the present invention.

A preferred group of compounds of formula (I) for the method according to the present invention comprises those compounds of a general formula (I) wherein the carbon atoms linked to the hydroxyl and amino groups have an absolute configuration (R).

Even more preferred compounds for use in the methods of the invention are the compounds of a general formula (I) wherein carbon atoms linked to the hydroxyl, amino, and phenoxy groups have an absolute configuration (R).

For the treatment or prophylaxis of depression the compounds of a general formula (I) may be administered orally, sublingually, transdermally, rectally, subcutaneously, intramuscularly, or intravenously.

The amount of active principle to be administered will depend, as usually, on the nature and severity of the pathological conditions to be treated as well as on the weight of the patient and the administration route.

In human beings the daily dosage typically varies between 0.01 and 30 mg/kg of body weight, and preferably between 0.01 and 10 mg/kg of body weight. Said daily dosage may be subdivided into 2, 3, or 4 administrations.

Preferably, for the treatment or prophylaxis of depression, the active principles (the compounds of a general formula (I) and their pharmaceutically acceptable metabolites or salts) are formulated in unit dosage forms containing from 0.1 to 1000 mg, more preferably from 0.5 to 800 mg, and most preferably from 0.8 to 500 mg of active principle in admixture with a pharmaceutical vehicle. Unit dosage forms suitable for oral administration include tablets, capsules, gel caps, powders, granules, and solutions or suspensions for oral use.

When a solid composition is prepared in the form of tablets, the main active ingredient is mixed with a pharmaceutical carrier such as gelatine, starch, lactose, magnesium stearate, talc, arabic gum, and the like. The tablets may be coated with sucrose or other appropriate materials or they may be treated so that their activity is extended or delayed and they continuously release a predetermined amount of active ingredient.

A preparation in the form of capsules is obtained by mixing the active ingredient with a diluent and filling the obtained mixture in soft or hard capsules.

A preparation in the form of a syrup or elixir or for the administration in drops may contain the active ingredient jointly with a sweetening agent, possibly acaloric, methylparaben and propylparaben as antiseptics, a flavoring agent and an appropriate dye.

Water-dispersible powders or granules may contain the active principle mixed with dispersing agents, wetting agents or suspending agents such as polyvinylpyrrolidone and the like, and with sweetening or flavouring agents as well.

For rectal administration, suppositories can be utilized, which are prepared with vehicles melting at rectal temperature, such as cocoa butter or polyethyleneglycols.

For sublingual administration microtablets or microcapsules can be prepared which, placed under the tongue, will rapidly dissolve. These compositions will generally contain the active ingredient in admixture with wetting and/or dispersing agents and optionally with absorption enhancers.

For transdermal administration, the use of polymeric diffusion matrices for the continuous and preferably sustained release of the active principle can be devised as well as the use of the active principle as a microemulsion with excipients adapted for contact with the skin.

For parenteral administration, aqueous suspensions, isotonic saline solutions, or sterile injectable solutions are used, which contain pharmaceutically compatible dispersing and/or wetting agents, for example propyleneglycol or butyleneglycol.

The active principle of a general formula (I) may also be formulated in the form of microcapsules, possibly with one or more carriers or additives.

The active principle of a general formula (I) may be administered as the free base or a pharmaceutically acceptable salt thereof, as such or complexed with a cyclodextrine, or even in admixture with or associated to other active principles.

By way of example, suitable pharmaceutical compositions may be prepared as follows:

Tablets containing Compound A as the active ingredient and having the composition—Compound A 5 mg, microcrystalline cellulose 40 mg, dried corn starch 40 mg, lactose 100 mg, and magnesium stearate 5 mg—are prepared by grinding the active ingredient to a particle size of 0.4 mm, passing it through a sieve with 0.4 mm openings, mixing the obtained ground active principle with the other ingredients and compressing the mixture to obtain tablets. Analogously, tablets containing 10 mg of active principle can be obtained.

By operating essentially as described above but using a metabolite of Compound A as the active principle, tablets with the following composition are prepared: metabolite of Compound A 50.0 mg, dried corn starch 100.0 mg, lactose 95.0 mg, talc 4.5 mg, magnesium stearate 0.5 mg.

By operating essentially as described above but using a maleic acid addition salt of Compound A as the active principle, tablets with the following composition are prepared: maleic acid addition salt of Compound A 60.0 mg, dried corn starch 120.0 mg, lactose 110.0 mg, talc 6.0 mg, magnesium stearate 0.6 mg.

10,000 capsules containing 20 mg each of active principle are prepared starting from the following ingredients: Compound A (200 g), microcrystalline cellulose (990 g), amorphous silica (10 g). The above ingredients are mixed together and then filled into hard gelatin capsules.

An aqueous sterile solution suitable for parenteral administration, as a mono-dose form, may have the following composition: Compound A 5 mg, sodium chloride 1 mg, distilled water q.s.p. 2 ml.

Examples of Finished Compositions

Compound A 0.5 mg, 5 mg, 50 mg and 100 mg Capsules are white opaque capsules in white 60 cc HDPE bottles with polypropylene caps. An exemplary composition is presented below:

Capsules

| | | Amount in mg/capsule | | | |
|---|---|---|---|---|---|
| Ingredient | Function | 0.5 mg | 5.0 mg | 50 mg | 100 mg |
| Compound A | Active | 0.5 | 5.0 | 50.0 | 100.0 |
| | ingredient | | | | |
| Lactose | Diluent | 184.3 | 163.8 | 161.0 | 170.1 |
| monohydrate, NF | | | | | |
| Colloidal silicon | Glidant | 0.2 | 0.2 | 0.25 | 0.32 |
| dioxide, NF | | | | | |
| Sodium starch | Disintegrant | 10.0 | 10.0 | 12.5 | 16.0 |
| glycolate, NF | | | | | |
| Poloxamer 407, | Surfactant | 4.0 | 20.0 | 25.0 | 32.0 |
| NF | | | | | |
| Magnesium | Lubricant | 1.0 | 1.0 | 1.25 | 1.6 |
| stearate, NF | | | | | |
| Gelatin capsule, | Encapsulation | NA | NA | NA | NA |
| hard | | | | | |

Typical Batch Formula

Batch sizes may vary. The formula for a typical batch of 4000 capsules manufactured by the process described herein is listed below:

| | 0.5 mg | 5 mg | 50 mg | 100 mg |
|---|---|---|---|---|
| Ingredient | Capsule | Capsule | Capsule | Capsule |
| Compound A | 2.0 g | 20 g | 200 g | 400 g |
| Lactose monohydrate, NF | 737.2 g | 655.2 g | 644.0 g | 680.4 g |
| Colloidal silicon dioxide, | 0.8 g | 0.8 g | 1.0 g | 1.28 g |
| NF | | | | |
| Sodium starch glycolate, | 40.0 g | 40.0 g | 50.0 g | 64.0 g |
| NF | | | | |
| Poloxamer 407, NF | 16.0 g | 80.0 g | 100.0 g | 128.0 g |
| Magnesium stearate, NF | 4.0 g | 4.0 g | 5.0 g | 6.4 g |
| Total | 800 g | 800 g | 1,000 g | 1,280.1 g |

Method of Manufacture

The stepwise process for the manufacture of Compound A 0.5 mg, 5 mg, 50 mg and 100 mg Capsules is presented below:
1. Through a mesh screen dispense the specified quantities of lactose, sodium starch glycolate, poloxamer, magnesium stearate, colloidal silicon dioxide and Compound A into separate polyethylene lined containers.
2. Add the specified quantities of lactose and colloidal silicon dioxide to a polyethylene lined container and label as PRE-MIX #1.
3. For 0.5 mg capsule strength:
   3.1. Add the specified quantities of lactose, poloxamer, and sodium starch glycolate to a polyethylene lined container and label as PRE-MIX #2.
   3.2. Add the specified quantities of lactose and Compound A to a polyethylene lined container and label as PRE-MIX #3.
   3.3. Add PRE-MIX #1 and PRE-MIX#3 to a V-Shell blender and mix for 2 minutes.
   3.4. Add PRE-MIX #2 to the mixture and blend for 2 minutes.
   3.5. Add the remaining lactose to the mixture and blend for 15 minutes.
4. For 5 mg, 50 mg and 100 mg capsule strengths:
   4.1. Add the specified quantities of lactose, poloxamer, sodium starch glycolate and Compound A to a polyethylene lined container and label as PRE-MIX #2.
   4.2. Add PRE-MIX #1 and PRE-MIX#2 to a V-Shell blender and mix for 20 minutes.
5. Add the magnesium stearate to the mixture and blend for 3 minutes.

6. Encapsulate the blend with the specified capsule.

| | 0.5 mg | | 50 mg | 100 mg |
|---|---|---|---|---|
| Ingredient | Capsule | 5 mg Capsule | Capsule | Capsule |
| PRE-MIX #1 | | | | |
| Lactose | 73.7 g | 131.0 g | 128.8 g | 136.1 g |
| Colloidal Silicon Dioxide | 0.8 g | 0.8 g | 1.0 g | 1.28 g |
| PRE-MIX #2 | | | | |
| Lactose | 147.4 g | 524.2 g | 515.2 g | 544.3 g |
| Poloxamer | 16.0 g | 80.0 g | 100.0 g | 128.0 g |
| Sodium Starch Glycolate | 40.0 g | 40.0 g | 50.0 g | 64.0 g |
| Compound A | NA | 20 g | 200 g | 400 g |
| PRE-MIX #3 | | | | |
| Lactose | 10.0 g | NA | NA | NA |
| Compound A | 2.0 g | NA | NA | NA |
| Lactose | 506.0 g | NA | NA | NA |
| Magnesium Stearate | 4.0 g | 4.0 g | 5.0 g | 6.4 g |
| Capsule Size | 3 | 3 | 2 | 1 |

Manufacturing Flow Chart
0.5 mg Strength Capsules 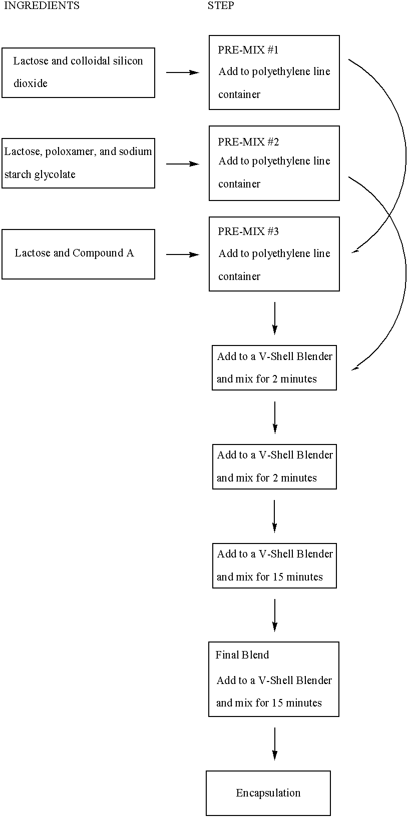
5 mg, 50 mg and 100 mg Strength Capsules

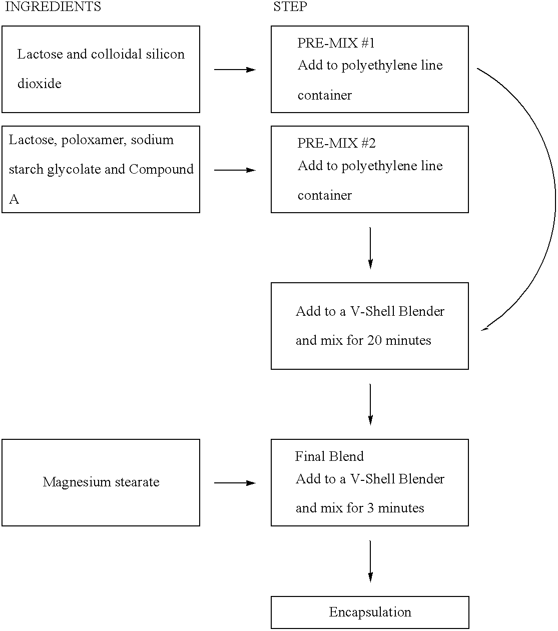

Preferred embodiments of the invention have been described above for the sake of illustration and should not be used to construe the invention in a limiting way.

What is claimed is:

1. A method of treatment or prophylaxis of depression which comprises administering to a mammal in need thereof a therapeutically or prophylactically effective amount of at least one compound of a general formula (I) 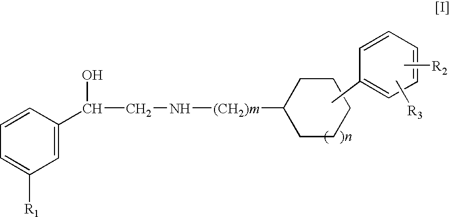
in which R₁ represents hydrogen or halogen; R₂ represents hydrogen, hydroxy, lower alkoxy, lower alkoxy substituted with one or two lower alkoxycarbonyl or carboxy groups, lower alkoxy substituted with lower alkylaminocarbonyl which may be substituted with lower alkoxy, lower alkoxy substituted with cyclic aminocarbonyl of 4 to 6 carbon atoms, lower alkoxycarbonyl or carboxy; R₃ represents hydrogen, hydroxy, lower alkoxy or lower alkoxy substituted with one or two lower alkoxycarbonyl or carboxy groups; R₂ and R₃ may be bonded to each other to form methylenedioxy substituted with carboxy or lower alkoxycarbonyl; and m and n are 0 or 1, and their pharmacologically acceptable metabolites and salts.

2. The method of claim 1 in which in which R₁ represents halogen.

3. The method of claim 1 in which R₂ represents lower alkoxy substituted with one or two lower alkoxycarbonyl groups.

4. The method of claim 1 in which R₃ represents hydrogen.

5. The method of claim 1 in which m is 0 and n is 1.

6. The method of claim 1 in which the at least one compound of a general formula (I) is ethyl 3-[(1R,3R)-3-[(2R)-2-(3-chlorophenyl)-2-hydroxyethylamino]cyclohexyl]phenoxyacetate or a pharmaceutically acceptable metabolite or salt thereof.

7. The method of claim 6 in which the pharmaceutically acceptable metabolite thereof is the ethyl ester hydrolysis product, 3-[(1R,3R)-3-[(2R)-2-(3-chlorophenyl)-2-hydroxyethyl-ammonium]cyclohexyl]phenoxyacetate or a pharmaceutically acceptable salt thereof.

8. The method of claim 6 in which the pharmaceutically acceptable salt thereof is a maleate salt.

9. The method of claim 1 in which the at least one compound of a general formula (I) or a pharmaceutically acceptable metabolite or salt thereof is administered in a daily dosage ranging from about 0.01 to about 30 mg/kg.

10. The method of claim 9 in which the daily dosage ranges from about 0.01 to about 10 mg/kg.

11. A method of treatment or prophylaxis of depression which comprises administering to a mammal in need thereof a therapeutically or prophylactically effective amount of at least one phenylethanolaminocyclohexylphenoxyacetic acid ester compound or a pharmacologically acceptable metabolite or salt thereof.

12. The method of claim 11 in which the at least one compound exhibits selective stimulating activity and binding affinity for β₃ adrenergic receptors.

13. The method of claim 11 in which one, two, or all three chiral centers of the at least one phenylethanolaminocyclohexylphenoxyacetic acid ester compound have a stereochemical configuration (R).

14. The method of claim 1, wherein the mammal is a human.
